(19) United States
(12) Patent Application Publication
(10) Pub. No.: US 2010/0068162 A1
(43) Pub. Date: Mar. 18, 2010

(54) Compositions Having Silicone Properties

(75) Inventors: Stephen Greenberg, Mahwah, NJ (US); Jed A. Riemer, Scarsdale, NY (US)

Correspondence Address: MARSHALL, GERSTEIN & BORUN LLP, 233 SOUTH WACKER DRIVE, 6300 SEARS TOWER, CHICAGO, IL, 60606-6357, US

(73) Assignee: LIPO CHEMICALS INC., Paterson, NJ (US)

(21) Appl. No.: 12/561,465

(22) Filed: Sep. 17, 2009

Related U.S. Application Data
(60) Provisional application No. 61/098,077, filed on Sep. 18, 2008.

(51) Int. Cl. A61K 8/85; A61Q 17/04; A61Q 1/02; A61Q 1/06; A61Q 15/00; A61Q 1/10; A61P 17/10; A61K 31/765; A61Q 5/02; A61Q 19/02

(52) U.S. Cl. 424/59; 424/62; 424/63; 424/64; 424/65; 424/70.7; 424/78.02; 424/78.03; 510/119

(57) ABSTRACT

Silicone-free, carbon-based replacements for volatile silicones, such as cyclomethicones, are disclosed. The compositions can be used as is or in dermatological products.

### CROSS-REFERENCE TO RELATED APPLICATIONS

This application claims the benefit of U.S. provisional patent application No. 61/098,077, filed Sep. 18, 2008, incorporated by reference herein in its entirety.

### FIELD OF THE INVENTION

The present invention relates to silicone-free, carbon-based substitutes for silicones, particularly volatile silicones, like cyclomethicone. The present invention also relates to dermatological compositions containing silicone-free compositions of the present invention.

### BACKGROUND OF THE INVENTION

Cyclomethicones are cyclic tetrameric, pentameric, and hexameric dimethicone compounds that are liquid at room temperature. Cyclomethicones also have a sufficiently low boiling point to be useful in dermatological compositions because of their ability to rapidly evaporate from the skin and hair. The cyclomethicones rapidly became a delivery vehicle or emollient of choice for many products including hair care products, such as shampoos and conditioners, antiperspirants and deodorants, and skin and body lotions. Recently, the use of the cyclomethicones has come under increased scrutiny because of environmental and toxicological issues.

The benefits of using cyclomethicone in skin care products include imparting a soft and silky feeling to the skin, evaporation at room temperature, an excellent spreading quality, leaving no oily residue or buildup, detackification, a nongreasy feel, compatibly with a wide range of cosmetic ingredients, a low surface tension, a transient emolliency, and improved rub in and spread. In hair care, the benefits include transient conditioning, lack of build up, and improved wet comb. In addition to the above positive attributes, cyclomethicone has a negative property of defatting and drying out the skin.

Due to growing safety concerns with respect to cyclomethicones, a need exists for compositions having similar physical and sensory properties that can be used as a substitute for cyclomethicone. The art therefore has been seeking non-silicone replacements for cyclomethicone, and other volatile silicones, that exhibit the benefits of volatile silicones, while avoiding the disadvantages associated with silicones.

### SUMMARY OF THE INVENTION

Surprisingly, it has been discovered that a mixture a volatile hydrocarbon, a non-volatile hydrocarbon, and a non-volatile polymeric ester can be used to mimic the properties of cyclomethicone in dermatological products, such as hair care products, skin care products, and cosmetics. A present non-silicone composition has an additional advantage over cyclomethicone in that it does not defat and dry the skin.

The present invention therefore relates to novel compositions for use as is or in dermatological products to provide the properties of hair conditioning, light emolliency, and lubricity without oiliness. The invention particularly relates to compositions comprising a volatile hydrocarbon, a nonvolatile hydrocarbon, and a polymeric ester, and the use of the compositions in dermatological compositions, such as hair care compositions, skin care compositions, cosmetic products, and topical medicaments.

In one aspect of the present invention, the compositions are used as a substitute for cyclomethicones that are added to a variety of dermatological compositions for their solvency, light emolliency (prior to evaporation), and volatility. It unexpectedly has been discovered that the inclusion of a present composition in a dermatological composition provides the properties of light emolliency, lubricity without oiliness, and a lightness similar to that of cyclomethicone and other cyclic silicones. The present compositions are advantageously used as substitutes for cyclomethicone because they provide a similar “initial feel” (i.e., when the compound is placed on the skin until about 5 minutes after being placed on the skin) and a superior “lasting feel” with respect to emolliency, as well as moisturizing characteristics, without the disadvantages associated with cyclomethicones, and in particular, environmental and toxicological concerns and defatting and drying of the skin.

Therefore, one aspect of the present invention to provide novel compositions that exhibit characteristics equal to, or essentially equal to, cyclomethicone with respect to lubricity and “after feel”, and that evaporate from the skin or hair, without exhibiting the problems and disadvantages associated with cyclomethicone.

It is another aspect of the invention to provide dermatological compositions formulated using a present composition.

These and other aspects of the present invention will become apparent from the following detailed description of the preferred embodiments of the invention.

### DETAILED DESCRIPTION OF THE PREFERRED EMBODIMENTS

The present invention is directed to compositions that can be used as a substitute for a cyclomethicone. The compositions are carbon-based and silicone-free. A present composition comprises (a) about 30% to about 90%, by weight, of a volatile hydrocarbon, (b) about 10% to about 70%, by weight, of a nonvolatile hydrocarbon, and (c) about 0.1% to about 10%, by weight, of a polymeric ester.

In accordance with the present invention, the composition comprises about 30% to about 90%, by weight, and preferably about 40% to about 80%, by weight, of a volatile hydrocarbon. To achieve the full advantage of the present invention, the composition comprises about 30% to about 70%, by weight, of a volatile hydrocarbon.

The volatile hydrocarbon is aliphatic, typically has about 8 to about 24 carbon atoms, and a sufficient volatility to slowly volatize from the hair and skin to prevent a residual buildup of hydrocarbon on dry hair and skin. The volatile hydrocarbon provides benefits such as lubrication and wet hair conditioning, for example.

Preferred volatile hydrocarbon compounds are aliphatic hydrocarbons including about 8 to about 24 carbon atoms, and a boiling point of about 100° C. to about 300° C. The volatile hydrocarbon therefore can be a straight chain hydrocarbon, such as a C₈ hydrocarbon (octane) or a C₂₀ hydrocarbon (eicosoane).

The volatile hydrocarbon also can be any structural isomer of a C₈ through C₂₄ hydrocarbon. In particular, the volatile hydrocarbon can be any aliphatic C₈ through C₂₄ hydrocarbon, straight chain or branched, i.e., C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, and any mixtures thereof.

Exemplary volatile hydrocarbons are depicted in general structural formula (I), wherein n ranges from 2 to 5.

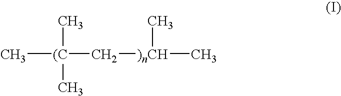

Especially preferred volatile hydrocarbons include octane, isodecane, isododecdane, dicosane, isoeicosane, dicosane, isohexadecane, hydrogenated didecene, and mixtures thereof.

Other useful volatile hydrocarbons include, but are not limited to, C7-8 isoparaffin, C8-9 isoparaffin, C9-11 isoparaffin, C9-13 isoparaffin, C9-14 isoparaffin, C10-11 isoparaffin, C10-13 isoparaffin, C11-12 isoparaffin, C11-13 isoparaffin, C12-14 isoparaffin, C13-14 isoparaffin, C13-16 isoparaffin, and mixtures thereof.

In addition to the volatile hydrocarbon, a present composition contains about 10% to about 70%, by weight, and preferably about 20% to about 60%, by weight, of a nonvolatile hydrocarbon. To achieve the full advantages of the present invention, the composition comprises about 30% to about 50%, by weight, of a nonvolatile hydrocarbon.

The nonvolatile hydrocarbon has a sufficient molecular weight and sufficiently high boiling point to remain on the skin or hair after application. The nonvolatile hydrocarbon imparts an improved feel, like softness, to treated hair and skin. The nonvolatile hydrocarbon imparts improved combining when applied to hair.

Preferably, the nonvolatile hydrocarbon is a hydrogenated polyalkene that is substantially free of phenyl groups. Still more preferably, the hydrogenated polyalkenes is a hydrogenated polyisoalkene. The term “polyisoalkene” refers to a polyalkene that includes a skeletal isocarbon in the repeating unit of the polyalkene. A skeletal “isocarbon” is a skeletal carbon atom that is chemically bound to three carbon atoms in the repeating unit. Preferably, substantially all the olefinic bonds of the polyolefin are hydrogenated to produce a fully saturated polyolefin. Fully saturated herein means at least 90% of the free olefinic groups of the polyolefin are saturated by hydrogenation.

In certain embodiments, the nonvolatile hydrocarbon comprises hydrogenated didecene, hydrogenated polydecene, hydrogenated polyisobutene, or a mixture thereof.

In addition to the volatile hydrocarbon and nonvolatile hydrocarbon, a present composition comprises a polymeric ester. The polymeric ester provides an improved skin or hair feel. The composition comprises about 0.1% to about 10%, by weight, of the polymeric ester, and preferably about 0.3% to about 5%, by weight, of the ester. To achieve the full advantages of the present invention, the composition comprises about 0.5% to about 2.5%, by weight, of the polymeric ester.

One preferred polymeric ester comprises bis-behenyl/isostearyl/phytosteryl dimer dilinoleyl dimer dioleate, available commercially as Plandool-G, manufactured by Nippon Fine Chemicals, Osaka, Japan and distributed by Lipo Chemicals Inc., Paterson, N.J. Other useful polymeric esters are phytostearyl/isostearyl/cetyl/stearyl/behenyl dimer dilinoleate, bisbehenyl/phytostearyl dimer dilinoleate, dimer dilinoleyl dimer dilinoleate, and any mixture thereof.

In one embodiment, a present composition comprises:

(a) 60%, by weight, isododecane;

(b) 39%, by weight, hydrogenated polydecene; and

(c) 1%, by weight, Plandool-G.

A composition of the present invention is used as is as a topically applied composition, or is incorporated into dermatological compositions as a substitute for a cyclomethicone. A dermatological composition comprising a silicone-free composition of the present invention imparts essentially the same properties to skin and hair as an identical composition containing a cyclomethicone as opposed to a composition of the present invention.

Unexpectedly, a present composition exhibits parity with a cyclomethicone in terms of lubricity (“dryness”, i.e., non-oily) and “after feel” (i.e., the present compositions are not felt on the skin after a short period of time), and with respect to lasting emolliency and compatibility with the skin, as well as moisturizing characteristics. This mixture is similar to cyclomethicone in properties such as skin feel, drying time, viscosity, spreadability, and after-feel. Thus, the present compositions demonstrate a parity with cyclomethicones with respect to features that are desirable, and provide advantages compared to cyclomethicones with respect to those features which are less desirable, i.e., environmental and toxicological concerns and defatting and drying of the skin. Thus, compositions of the present invention can be used as cyclomethicone substitutes exhibiting the same or more favorable properties than cyclomethicones in dermatological compositions.

A composition of the present invention can be used as is or incorporated into a dermatological composition in an amount of about 0.1% to about 95%, by weight, and typically about 0.5% to about 50%, by weight, of the dermatological composition. The dermatological composition can be a water-in-oil emulsion, an oil-in-water emulsion, a solution, a dispersion, a cream, a solid, a gel, or a lotion, for example.

A silicone-free composition of the present invention is useful in all classes of dermatological compositions, e.g., personal care, cosmetic, and pharmaceutical compositions. The dermatological compositions provide a silicone-like feel and conditioning in the absence of a silicone compound. The dermatological compositions for treating skin or hair can be formulated with topically-applied active compounds.

In accordance with an important feature of the present invention, a topically-applied compound for providing a cosmetic or therapeutic effect can be any of a wide variety of compounds, either water soluble or oil soluble.

Topically-applied active compounds, such as hormones, analgesics, anesthetics, sun screens, skin whiteners, antiacne agents, antibacterial agents, antifungal agents, botanical extracts, pharmaceuticals, minerals, plant extracts, concentrates of plant extracts, exfoliants, emollients, moisturizers, skin protectants, humectants, silicones, skin soothing ingredients, colorants, perfumes, and like can be included in the dermatological composition. The quantities of such active compounds present in the dermatological composition are sufficient to perform their intended function.

More particularly, such a topically applied active compound can be one of, or a mixture of, a cosmetic compound, a medicinally active compound, a compound used in cosmetics or personal care, or any other compound that is useful upon topical application to the skin. Such topically active agents include, but are not limited to, skin-care compounds, plant extracts, antioxidants, insect repellants, counterirritants, vitamins, steroids, antibacterial compounds, antifungal compounds, antiinflammatory compounds, topical anesthetics, sunscreens, optical brighteners, and other cosmetic and medicinal topically effective compounds.

For example, a skin conditioner can be the topically applied compound. Skin conditioning agents include, but are not limited to, humectants, such a fructose, glucose, glycerin, propylene glycol, glycereth-26, mannitol, pyrrolidone carboxylic acid, hydrolyzed lecithin, coco-betaine, cysteine hydrochloride, glucamine, sodium gluconate, potassium aspartate, oleyl betaine, thiamine hydrochloride, sodium laureth sulfate, sodium hyaluronate, hydrolyzed proteins, hydrolyzed keratin, amino acids, amine oxides, water-soluble derivatives of vitamins A, E, and D, selenium and derivatives thereof, amino-functional silicones, ethoxylated glycerin, alpha-hydroxy acids and salts thereof, fatty oil derivatives, such as PEG-24 hydrogenated lanolin, beta-hydroxy acids and salts thereof (e.g., glycolic acid, lactic acid, and salicylic acid), and mixtures thereof. Numerous other skin conditioners are listed in the CTFA Cosmetic Ingredient Handbook, First Ed., J. Nikotakis, ed., The Cosmetic, Toiletry and Fragrance Association (1988), (hereafter CTFA Handbook), pages 79-84, incorporated herein by reference.

The skin conditioner also can be a water-insoluble ester having at least 10 carbon atoms, and preferably 10 to about 32 carbon atoms. Suitable esters include those comprising an aliphatic alcohol having about eight to about twenty carbon atoms and an aliphatic or aromatic carboxylic acid including from two to about twelve carbon atoms, or conversely, an aliphatic alcohol having two to about twelve carbon atoms with an aliphatic or aromatic carboxylic acid including about eight to about twenty carbon atoms. The ester is either straight-chained or branched. Suitable esters, therefore, include, for example, but are not limited to:

(a) aliphatic monohydric alcohol esters, including, but not limited to: myristyl propionate, isopropyl isostearate, isopropyl myristate, isopropyl palmitate, cetyl acetate, cetyl propionate, cetyl stearate, isodecyl neopentanoate, cetyl octanoate, isocetyl stearate;

(b) aliphatic di- and tri-esters of polycarboxylic acid, including, but not limited to: di isopropyl adipate, diisostearyl fumarate, dioctyl adipate, and triisostearyl citrate;

(c) aliphatic polyhydric alcohol esters, including, but not limited to: propylene glycol dipelargonate;

(d) aliphatic esters of aromatic acids, including, but not limited to: C₁₂-C₁₅ alcohol esters of benzoic acid, octyl salicylate, sucrose benzoate, and dioctyl phthalate.

Numerous other esters are listed in the CTFA Handbook, at pages 24 through 26, incorporated herein by reference.

The topically applied compound also can be retinoic acid or a retinol derivative.

The topically applied compound further can be an antioxidant or an optical brightener, like a distyrylbiphenyl derivative, stilbene or a stilbene derivative, a pyralozine derivative, or a coumarin derivative. Optical brighteners useful as the topically applied compound can be any compound capable of absorbing an invisible UV portion of the daylight spectrum, and converting this energy into the longer visible wavelength portion of the spectrum. The optical brightener is colorless on the substrate, and does not absorb energy in the visible part of the spectrum. The optical brightener typically is a derivative of stilbene or 4,4′-diaminostilbene, biphenyl, a 5-membered heterocycle, e.g., triazole, oxazole, or imidazole, or a 6-membered heterocycle, e.g., a coumarin, a naphthalamide, or an s-triazine.

The optical brighteners are available under a variety of tradenames, such as TINOPAL®, LEUCOPHOR®, and CALCOFLUOR®. Specific fluorescent compounds include, but are not limited to, TINOPAL® 5BM, CALCOFLUOR® CG, and LEUCOPHOR® BSB.

In addition, other compounds can be included in a dermatological composition as the topically active compound in an amount sufficient to perform their intended function. For example, sunscreen compounds such as benzophenone-3, tannic acid, uric acids, quinine salts, dihydroxy naphtholic acid, an anthranilate, p-aminobenzoic acid, phenylbenzimidazole sulfonic acid, PEG-25, or p-aminobenzoic acid can be used as the topically applied compound. Further, sunscreen compounds such as dioxybenzone, ethyl 4-[bis(hydroxypropyl)]aminobenzoate, glyceryl aminobenzoate, homosalate, methyl anthranilate, octocrylene, octyl methoxycinnamate, octyl salicylate, oxybenzone, padimate O, red petrolatum, titanium dioxide, 4-menthylbenzylidene camphor, benzophenone-1, benzophenone-2, benzophenone-6, benzophenone-12, isopropyl dibenzoyl methane, butyl methoxydibenzoylmethane, zotocrylene, or zinc oxide can be used as the topically applied compound. Other sunscreen compounds are listed in CTFA Handbook, pages 86 and 87, incorporated herein by reference.

Similarly, topically applied drugs, like antifungal compounds, antibacterial compounds, antiinflammatory compounds, topical anesthetics, skin rash, skin disease, and dermatitis medications, and antiitch and irritation-reducing compounds can be used as the active agent in the compositions of the present invention. For example, analgesics such as benzocaine, dyclonine hydrochloride, aloe vera, and the like; anesthetics such as butamben picrate, lidocaine hydrochloride, xylocaine, and the like; antibacterials and antiseptics, such as povidone-iodine, polymyxin b sulfate-bacitracin, zinc-neomycin sulfate-hydrocortisone, chloramphenicol, ethylbenzethonium chloride, erythromycin, and the like; antiparasitics, such as lindane; essentially all dermatologicals, like acne preparations, such as benzoyl peroxide, erythromycin benzoyl peroxide, clindamycin phosphate, 5,7-dichloro-8-hydroxyquinoline, and the like; antiinflammatory agents, such as alclometasone dipropionate, betamethasone valerate, and the like; burn relief ointments, such as o-amino-p-toluenesulfonamide monoacetate, and the like; depigmenting agents, such as monobenzone; dermatitis relief agents, such as the active steroid amcinonide, diflorasone diacetate, hydrocortisone, and the like; emollients and moisturizers, such as mineral oil, PEG-4 dilaurate, lanolin oil, petrolatum, mineral wax, and the like; fungicides, such as butocouazole nitrate, haloprogin, clotrimazole, and the like; herpes treatment drugs, such as O-[(2-hydroxymethyl)-methyl]guanine; pruritic medications, such as alclometasone dipropionate, betamethasone valerate, isopropyl myristate MSD, and the like; psoriasis, seborrhea, and scabicide agents, such as anthralin, methoxsalen, coal tar, and the like; steroids, such as 2-(acetyloxy)-9-fluoro-1′,2′,3′,4′-tetrahydro-11-hydroxypregna-1,4-dieno-[16,17-b]naphthalene-3,20-dione and 21-chloro-9-fluoro-1′,2′,3′,4′-tetrahydro-11b-hydroxypregna-1,4-dieno-[16,17-b]naphthalene-3,20-dione. Any other medication capable of topical administration, like skin protectants, such as allantoin, also can be incorporated in a composition of the present invention in an amount sufficient to perform its intended function. Other topically applied compounds are listed in Remington's Pharmaceutical Sciences, 17th Ed., Mack Publishing Co., Easton, Pa. (1985), pages 773-791 and pages 1054-1058 (hereinafter Remington's), incorporated herein by reference.

The topically active compound also can be a plant extract on a natural oil. Numerous plant extracts are available from Lipo Chemicals, Inc. Paterson, N.J. Nonlimiting plant extracts are those obtained from alfalfa, aloe vera, amla fruit, angelica root, anise seed, apple, apricot, artichoke leaf, asparagus root, banana, barberry, barley sprout, bee pollen, beet leaf, bilberry fruit, birch leaf, bitter melon, black currant leaf, black pepper, black walnut, blueberry, blackberry, burdock, carrot, cayenne, celery seed, cherry, chickwood, cola nut, corn silk, cranberry, dandelion root, elderberry, eucalyptus leaf, flax oil powder, ginger root, gingko leaf, ginseng, goldenrod, goldenseal, grape, grapefruit, guava, hibiscus, juniper, kiwi, kudzu, lemon, licorice root, lime, malt, marigold, myrrh, olive leaf, orange fruit, orange peel, oregano, papaya fruit, papaya leaf, passion fruit, peach, pear, pine bark, plum, pomegranate, prune, raspberry, rhubarb root, rosemary leaf, sage leaf, spearmint leaf, St. John's wart, strawberry, sweet cloves, tangerine, violet herb, watercress, watermelon, willow bark, wintergreen leaf, witch hazel bark, yohimbe, and yucca root. An example of a natural oil is rice bran oil.

A composition of the present invention is prepared by admixing the volatile hydrocarbon, nonvolatile hydrocarbon, and polymeric ester. The present compositions can be admixed with other ingredients traditionally included in dermatological compositions. These ingredients include, but are not limited to, dyes, fragrances, preservatives, antioxidants, detackifying agents, antiperspirant actives, emollients, emulsifiers, surfactants, water, alcohols, common organic solvents, conditions, deodorants, vegetable oils, dispersants, and similar types of compounds. The ingredients are included in the composition in an amount sufficient to perform their intended function.

One or more of the following additional ingredients typically are included in a dermatological composition. Each of these ingredients, and any other ingredient, is present in a sufficient amount to perform its intended function together with a silicone-free composition of the present invention.

For example, a dermatological composition, in addition to, a present composition, can contain a surfactant. The surfactant can be an anionic surfactant, a cationic surfactant, a nonionic surfactant, or a compatible mixture of surfactants. The surfactant also can be an ampholytic or amphoteric surfactant, which have anionic or cationic properties depending upon the pH of the composition.

Examples of anionic surfactants include, without limitation, soaps, alkyl sulfates, anionic acyl sarcosinates, methyl acyl taurates, N-acyl glutamates, acyl isethionates, alkyl phosphate esters, ethoxylated alkyl phosphate esters, alkyl sulfosuccinates, trideceth sulfates, protein condensates, mixtures of ethoxylated alkyl sulfates, and the like. Examples of anionic nonsoap surfactants include, without limitation, the alkali metal salts of an organic sulfate having an alkyl radical containing about 8 to about 22 carbon atoms and a sulfonic acid or sulfuric acid ester radical. Examples of zwitterionic surfactants include, without limitation, derivatives of aliphatic quaternary ammonium, phosphonium, and sulfonium compounds, in which the aliphatic radicals can be straight chain or branched and wherein one of the aliphatic substituents contains an anionic water-solubilizing group, e.g., carboxyl, sulfonate, sulfate, phosphate, or phosphonate. Examples of amphoteric surfactants include, without limitation, derivatives of aliphatic secondary and tertiary amines in which the aliphatic radical can be straight chain or branched and wherein one of the aliphatic substituents contains about 8 to about 18 carbon atoms and one contains an anionic water solubilizing group, e.g., carboxyl, sulfonate, sulfate, phosphate, or phosphonate. Examples of cationic surfactants include, without limitation, stearyldimethylbenzyl ammonium chloride; dodecyltrimethyl ammonium chloride; nonylbenzylethyldimethyl ammonium nitrate; and tetradecylpyridinium bromide. Nonionic surfactants include, without limitation, compounds produced by the condensation of ethylene oxide groups with an organic hydrophobic compound, which may be aliphatic or alkyl aromatic in nature, for example, the polyethylene oxide condensates of alkyl phenols.

The dermatological composition also can contain a hydrotrope. A hydrotrope is a compound that has an ability to enhance the water solubility of other compounds. Specific examples of hydrotropes include, but are not limited to, sodium cumene sulfonate, ammonium cumene sulfonate, ammonium xylene sulfonate, potassium toluene sulfonate, sodium toluene sulfonate, sodium xylene sulfonate, toluene sulfonic acid, and xylene sulfonic acid. Other useful hydrotropes include sodium polynaphthalene sulfonate, sodium polystyrene sulfonate, sodium methyl naphthalene sulfonate, sodium camphor sulfonate, and disodium succinate.

The dermatological composition also can contain an additional organic solvent. The solvent can be a water-soluble organic compound containing one to six, and typically one to three, hydroxyl groups, e.g., alcohols, diols, triols, and polyols. Specific examples of solvents include, but are not limited to, methanol, ethanol, isopropyl alcohol, n-butanol, n-propyl alcohol, ethylene glycol, propylene glycol, glycerol, diethylene glycol, dipropylene glycol, tripropylene glycol, hexylene glycol, butylene glycol, 1,2,6-hexanetriol, sorbitol, PEG-4, 1,5-pentanediol, similar hydroxyl-containing compounds, and mixtures thereof. The solvent also can be water or an aprotic solvent, e.g., dimethyl sulfoxide or tetrahydrofuran.

The dermatological composition also can contain a thickening or gelling agent. A thickening or gelling agent can be, for example, a polymer that is water soluble or that generates a colloidal solution in water. A thickening or gelling agent, therefore, can be, for example, polymers or copolymers unsaturated carboxylic acids or unsaturated esters, polysaccharide derivatives, gums, colloidal silicates, polyethylene glycols (PEG) and their derivatives, polyvinylpyrrolidones and their derivatives, polyacrylamides and their derivatives, polyacrylonitriles, hydrophilic silica gels, or mixtures thereof.

Specific thickening or gelling agents can be, for example, acrylic and/or methacrylic polymers or copolymers, vinylcarboxylic polymers, polyglyceryl acrylates or methacrylates, polyacrylamides derivatives, cellulose or starch derivatives, chitin derivatives, alginates, amino acids, ceramides, fatty acids, cholesterol and derivatives thereof, and other natural moisturizing compounds, hyaluronic acid and its salts, chonodroitin sulphates, xanthan, gellan. Rhamsan, karaya or guar gum, carob flour, and colloidal aluminum magnesium silicates of the montmorillonite type.

Additional thickening or gelling agents include vinylcarboxylic polymers sold under the tradename CARBOPOL® (Goodrich), acrylic acid/ethyl acrylate copolymers, acrylic acid/stearyl methacrylate copolymers, carboxymethylcellulose, hydroxymethylcellulose, hydroxypropylcellulose, microcrystalline cellulose, hydroxypropyl guar, colloidal hectorites, bentonites, and the like.

Other classes of optional ingredients included in the dermatological composition can be, but are not limited to, pH adjusters, chelating agents, preservatives, buffering agents, foam stabilizers, opacifiers, and similar classes of ingredients known to persons skilled in the art. Specific optional ingredients include inorganic phosphates, sulfates, and carbonates as buffering agents; EDTA and phosphates as chelating agents; and acids and bases as pH adjusters.

Nonlimiting examples of basic pH adjusters are ammonia; mono-, di-, and tri-alkyl amines; mono-, di-, and tri-alkanolamines; alkali metal and alkaline earth metal hydroxides; and mixtures thereof. Specific, nonlimiting examples of basic pH adjusters are ammonia; sodium, potassium, and lithium hydroxide; monoethanolamine; triethylamine; isopropanolamine; diethanolamine; and triethanolamine. Examples of acidic pH adjusters are the mineral acids and organic carboxylic acids. Nonlimiting examples of mineral acids are citric acid, hydrochloric acid, nitric acid, phosphoric acid, and sulfuric acid.

The silicone-free composition can be incorporated into dermatological compositions designed as cosmetic basecoats and undercoats, bath capsules, bath oils, bath tablets, bath salts, bath soaps, blushers, face, body, and hand creams and lotions, cosmetic foundations, hormone creams and lotions, leg and body paints, makeup bases, makeup fixatives, makeup products, moisturizing creams and lotions, night creams and lotions, paste masks, skin care products, skin fresheners, skin lighteners, tonics, dressings, wrinkle smoothing creams and lotions, hair shampoos, antiperspirants, deodorants, lipsticks, facial cosmetics, sunscreens, mascaras, eye liners, eye shadows, and similar dermatological compositions.

In particular embodiments, the silicone-free composition can be incorporated into lotions; makeup preparations, like makeup foundations; skin care preparations, like hand lotions, vanishing creams, night creams, sunscreens, body lotions, facial creams, clay masks, moisturizing lotions, makeup removers, antiacne preparations, antiaging preparations, and sebum control preparations; preparations, analgesic and cortisonal steroid creams and preparations; insect repellants; topical medicaments; and facial masks and revitalizers. The present compositions also can be incorporated into plasters, bandages, dressings, gauze pads, and similar articles.

The dermatological composition can be in the form of a solution, an oil-in-water emulsion, a water-in-oil emulsion, a gel, a microemulsion, a nanoemulsion, or other product form known in the skin care and dermatological arts. A present non-silicone composition also can be delivered from an encapsulated or nonencapsulated delivery system, a liposome, or other vesicle or lamellar delivery system. The dermatological composition can be, for example, a liquid, e.g., a solution, a gelled solution, or a suspension in an aqueous or oily medium; or a semi-liquid formulation, e.g., a cream, a gel, a paste, an ointment, a salve, a liposome, an emulsion, or a microemulsion.

1. A composition comprising about 30% to about 90%, by weight, of a volatile hydrocarbon, about 10% to about 70%, by weight, of a nonvolatile hydrocarbon, and about 0.1% to about 10%, by weight, of a polymeric ester.

2. The composition of claim 1 comprising about 40% to about 80%, by weight, of the volatile hydrocarbon, about 20% to about 60%, by weight, of the nonvolatile hydrocarbon, and about 0.3% to about 5%, by weight, of the polymeric ester.

3. The composition of claim 1 wherein the volatile hydrocarbon is a straight chain or branched aliphatic hydrocarbon containing about 8 to about 24 carbon atoms.

4. The composition of claim 3 wherein the volatile hydrocarbon comprises 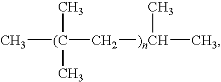
wherein n ranges from 2 to 5, octane, isodecane, isododecdane, dicosane, isoeicosane, dicosane, isohexadecane, hydrogenated didecene, C7-8 isoparaffin, C8-9 isoparaffin, C9-11 isoparaffin, C9-13 isoparaffin, C9-14 isoparaffin, C10-11 isoparaffin, C10-13 isoparaffin, C11-12 isoparaffin, C11-13 isoparaffin, C12-14 isoparaffin, C13-14 isoparaffin, C13-16 isoparaffin, and mixtures thereof.

5. The composition of claim 3 wherein the nonvolatile hydrocarbon comprises a hydrogentated polyalkene.

6. The composition of claim 5 wherein the hydrogenated polyalkene comprises a hydrogenated polyisoalkene.

7. The composition of claim 1 wherein the nonvolatile hydrocarbon comprises hydrogenated didecene, hydrogenated polydecene, hydrogenated polyisobutene, or a mixture thereof.

8. The composition of claim 1 wherein the polymeric ester is selected from the group consisting of bis-behenyl/isostearyl/phytosteryl dimer dilinoleyl dimer dilinoleate, phytosteryl/isostearyl/cetyl/stearyl/behenyl dimer dilinoleate, bis-behenyl/phytosteryl dimer dilinoleate, dimer dilinoleyl dimer dilinoleate, and mixtures thereof.

9. The composition of claim 1 comprising about 30% to about 70%, by weight, isododecane, 39%, by weight, hydrogenated polydecene, and about 0.5% to about 2.5%, by weight, bis-behenyl/isostearyl/phytosteryl dimer dilinoleyl dimer dilinoleate.

10. A dermatological composition comprising about 0.1% to about 95%, by weight, of a composition of claim 1.

11. The dermatological composition of claim 10 selected from the group consisting of cosmetic basecoats and undercoats, bath capsules, bath oils, bath tablets, bath salts, bath soaps, blushers, face, body, and hand creams and lotions, cosmetic foundations, hormone creams and lotions, leg and body paints, makeup bases, makeup fixatives, makeup products, moisturizing creams and lotions, night creams and lotions, paste masks, skin care products, skin fresheners, skin tighteners, tonics, dressings, wrinkle smoothing creams and lotions, hair shampoo, antiperspirants, deodorants, lipsticks, facial cosmetics, sunscreens, mascaras, eye liners, eye shadows, makeup preparations, makeup foundations, skin care preparations, hand lotions, vanishing creams, night creams, sunscreens, body lotions, facial creams, clay masks, moisturizing lotions, makeup removers, antiacne preparations, antiaging preparations, sebum control, preparations, analgesic and cortisonal steroid creams and preparations, insect repellants, topical medicaments, and facial masks and revitalizers.
